(19) United States
(12) Patent Application Publication
(10) Pub. No.: US 2006/0235424 A1
(43) Pub. Date: Oct. 19, 2006

(54) Implantable bone distraction device and method

(75) Inventors: Nicholas Gerard Vitale, Albany, NY (US); Arthur C. Donahue, East Greenbush, NY (US); Scott Andrew Wheeler, Ballston Lake, NY (US)

Correspondence Address: HESLIN ROTHENBERG FARLEY & MESITI PC, 5 COLUMBIA CIRCLE, ALBANY, NY, 12203, US

(73) Assignee: FOSTER-MILLER, INC., Albany, NY (US)

(21) Appl. No.: 11/394,502

(22) Filed: Mar. 31, 2006

Related U.S. Application Data
(60) Provisional application No. 60/667,389, filed on Apr. 1, 2005.

(51) Int. Cl. A61B 17/58

(52) U.S. Cl. 606/90

(57) ABSTRACT

A self-contained, implantable bone distraction device is provided. The device is controlled by a programmable microcontroller that communicates with the outside world wirelessly, for example, via radio frequency or infrared. The microcontroller can be instructed, for example, to initiate an immediate distraction, or to stop a distraction in progress. Nitinol wire is used in conjunction with a one-way clutch to cause a distraction increment. The length of the wire is maintained after deactivation mechanically. Optional sensors allow the monitoring of the amount of actual distraction or the distraction force experienced by the bone under distraction.

### CROSS-REFERENCE TO RELATED APPLICATIONS

This application claims priority under 35 U.S.C. §119 to U.S. Provisional Application No. 60/667,389, filed Apr. 1, 2005, which is herein incorporated by reference in its entirety.

### GOVERNMENT RIGHTS STATEMENT

This invention was made with U.S. Government support under Grant No. 5R44AR047257-03 from the National Institute of Health, National Heart, Lung and Blood Institute. The U.S. Government has certain rights in the invention.

### BACKGROUND OF THE INVENTION

1. Technical Field

The present invention generally relates to bone distraction. More particularly, the present invention relates to an implantable bone distraction device.

2. Background Information

Limb-shortening deformities and segmental defects occur as a result of trauma, surgical treatment of bone tumors and infections, and congenital or developmental deformities. Approximately 5,000 surgical procedures are performed each year in the United States to correct deformities by lengthening limbs. As many as 15,000 to 20,000 procedures are performed annually to replace or regenerate missing bone segments (>2.5 cm) in extremities. Extensive research has been performed to improve on existing methods and introduce new methods for bone transport and lengthening, as summarized below.

It has been reported that mature bone can be regenerated by gradual distraction of a healing fracture callus through a unique biologic process called distraction osteogenesis. However, bone lengthening and bone transport procedures originally used an external fixation device that is associated with other significant complications, usually related to the transfixing wires. These complications include wire site infection, pain, and restricted joint motion caused by the transfixation of skin, fascia, tendons and muscles. Union at the docking site, where bone ends finally meet in the center of the defect often is delayed, and frequently requires a small open grafting procedure. As a result, the overall morbidity and treatment time using this technique may exceed that associated with open bone grafting in many instances. Furthermore, the psychological stress associated with the prolonged treatment period (mean of about 300 days for a 10 cm defect) can lead to interruption or abortion of ongoing therapy. Uniplanar external fixators have been adapted to reduce some of these complications without severely compromising mechanical control of the involved segments. However, these newer devices have not eliminated the noted problems.

The problems stemming from external fixation can be eliminated by instead implanting a distraction device. However, efforts in that regard have not been entirely successful.

Thus, a need continues to exist for an improved, self-contained, implantable bone distraction device.

### SUMMARY OF THE INVENTION

Briefly, the present invention satisfies the need for an improved, self-contained, implantable bone distraction device by providing a programmable, battery-powered device. In one embodiment, the device communicates wirelessly to send information and/or receive commands or programming.

In accordance with the above, it is an object of the present invention to provide an implantable, programmable bone distraction device.

It is another object of the invention to provide an implantable bone distraction device that can communicate wirelessly.

It is yet another object of the present invention to provide an implantable bone distraction device that can be commanded to apply an immediate distraction and/or stop a distraction in progress.

It is still another object of the present invention to provide an implantable bone distraction device that can sense the actual distraction distance.

It is another object of the present invention to provide an implantable bone distraction device that can sense the distraction force experienced by the bone under distraction.

The present invention provides, in a first aspect, a bone distraction device. The device comprises a distraction driver for incrementally distracting bone and minimizing backlash, an actuator coupled to the distraction driver for actuating the distraction driver, and a microcontroller electrically coupled to the actuator for controlling the actuator. The device further comprises at least one of a wireless communications receiver electrically coupled to the microcontroller for receiving information and a wireless communications transmitter electrically coupled to the microcontroller for transmitting information, wherein the bone distraction device is implantable.

The present invention provides, in a second aspect, a system for bone distraction. The system comprises a bone distraction device, comprising a distraction driver for incrementally distracting bone and minimizing backlash, an actuator coupled to the distraction driver for actuating the distraction driver using a shape memory alloy, a microcontroller electrically coupled to the actuator for controlling the actuator, and a wireless communications transceiver electrically coupled to the microcontroller for transmitting and receiving information, wherein the bone distraction device is implantable. The system further comprises a wireless communications device for transmitting and receiving information from the wireless communications transceiver.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1 is a block diagram of one example of a distraction device in accordance with the present invention.

FIGS. 2A and 2B are a flow diagram of the programming for the microcontroller of FIG. 1.

FIG. 3 is a cross-sectional view of one example of a one-way roller clutch useful with the present invention.

FIGS. 4A-4D depict one example of a ratchet and pawl system useful with the present invention.

FIG. 5 depicts one example of a distraction device in accordance with the present invention.

FIG. 6 is a cross-sectional view of a portion of the distraction device of FIG. 5.

FIG. 7 is a more detailed view of a portion of the distraction device of FIG. 5.

FIG. 8 shows a portion of the distraction device of FIG. 5 in more detail.

FIG. 9 is a block diagram of a handheld computer useful in communicating with the distraction device of the present invention.

FIG. 10 is a cut-away view of a portion of the distraction device shown in FIG. 5.

FIG. 11 is a more detailed, cut-away view of the displacement sensor shown in FIG. 7.

FIG. 12 is a cut-away view of the SMA actuator of FIG. 8.

FIG. 13 depicts one example of a force sensor in accordance with the present invention.

FIG. 14 depicts a more detailed view of a portion of the one-way roller clutch of FIG. 3.

FIG. 15 depicts the various phase transformations of a shape memory alloy.

FIG. 16 is a graph of stress versus strain for the phase transformations depicted in FIG. 15.

FIG. 17 is a block diagram of a radio transceiver, one example of the wireless communications module in FIG. 1.

FIG. 18 is a block diagram of the analog circuitry in FIG. 1.

### DETAILED DESCRIPTION OF THE INVENTION

A self-contained, implantable bone distraction device is provided. In a preferred embodiment, the device is controlled by a programmable microcontroller that communicates with the outside world wirelessly, for example, via radio frequency or infrared. The microcontroller can be instructed, for example, to initiate an immediate distraction or to change the distraction time increment. A shape memory alloy (SMA) is actuated to cause a distraction increment. The length of the distraction cable between the device and the bone under distraction is maintained after deactuation via mechanical means. Sensors allow the monitoring of key parameters, depending on the application, for example, detecting the amount of actual distraction or the distraction force experienced by the bone under distraction. This information can be provided by the microcontroller to the outside world for monitoring.

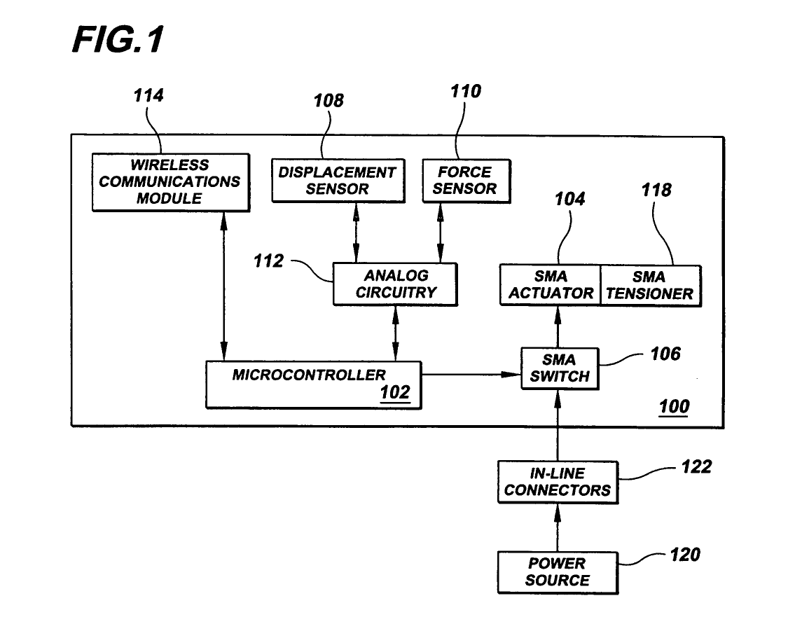

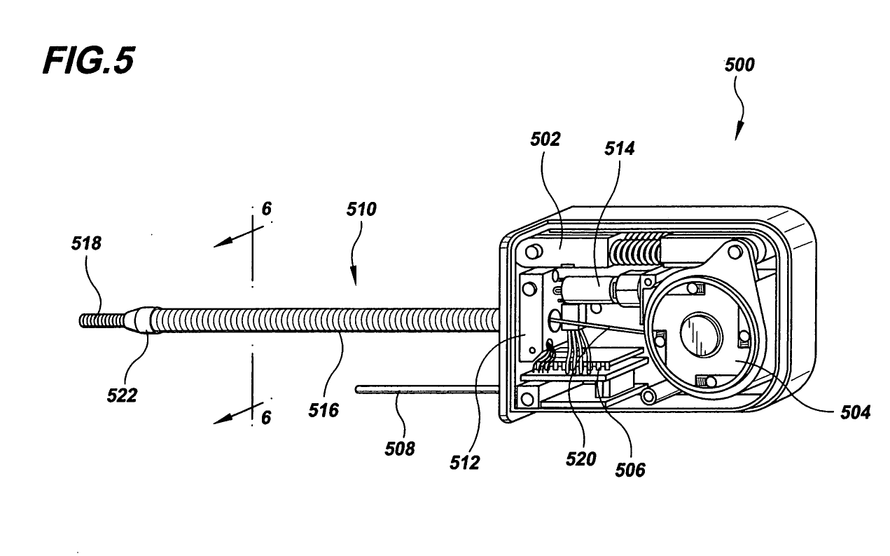

FIG. 1 is a block diagram of one example of a distraction device 100, in accordance with the present invention. The distraction device comprises a microcontroller 102, which is preferably programmable, for controlling the distraction device. The distraction device further comprises a SMA actuator 104, which will be explained in further detail below. A SMA switch 106, acting under instructions from microcontroller 102, causes the SMA actuator to turn on and off. The device also optionally comprises at least one sensor for sensing at least one characteristic of a distraction, for example, a displacement sensor 108 for sensing the actual amount of distraction obtained, and/or an optional force sensor 110 for sensing the amount of force experienced by the bone under distraction. Analog circuitry 112 interfaces displacement sensor 108 and force sensor 110 to microcontroller 102. A wireless communications module 114 provides communications between the distraction device and the outside world. A SMA tensioner 118 is coupled to the SMA actuator to maintain tension on the distraction cable (see FIG. 5) after distraction. Also shown in FIG. 1 is a DC power source 120 coupled to switch 106 through in-line connectors 122, for supplying power to the distraction device. In one example, the in-line connectors are a locking, polarized male-female pair that carry current to the SMA.

The microcontroller is the “brains” of the distraction device, controlling and coordinating the actions of all the other elements. Microcontroller 102 activates SMA actuator 104 by connecting the DC power source 120 to the SMA actuator 104 via SMA switch 106 for a time period determined by the value of the distraction time parameter. The microcontroller controls the time between actuations by the value of the distraction interval parameter. Preferably, the microcontroller is programmable, so that a clinician can alter the distraction time parameter and/or the distraction interval parameter where necessary or desired, e.g., based on medical data obtained during the course of treatment. Of course, electronics other than the microcontroller could also serve the purpose of the microcontroller, for example, a processor (microcomputer), programmable logic device, or dedicated circuitry, such as an application specific integrated circuit (ASIC), though an ASIC is generally not programmable. One example of a commercially available programmable microcontroller is Model PIC 16C57, a 4 MHz, 8-bit, RISC microcontroller manufactured by Microchip Technology, Inc, Chandler, Ariz.

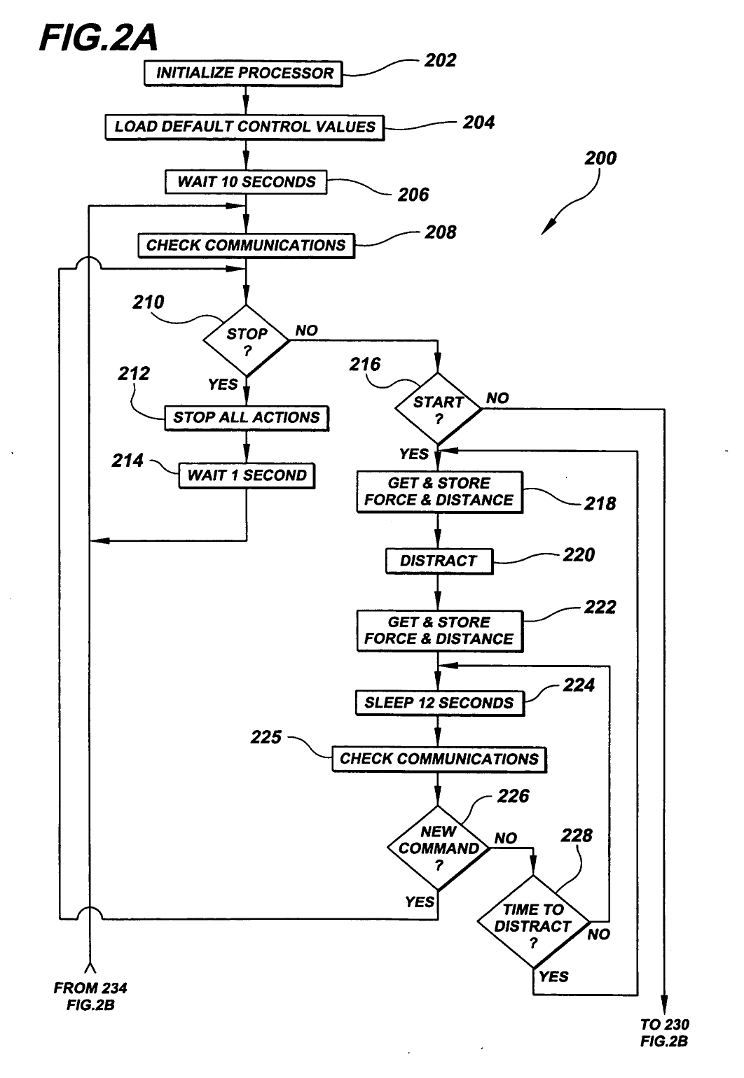

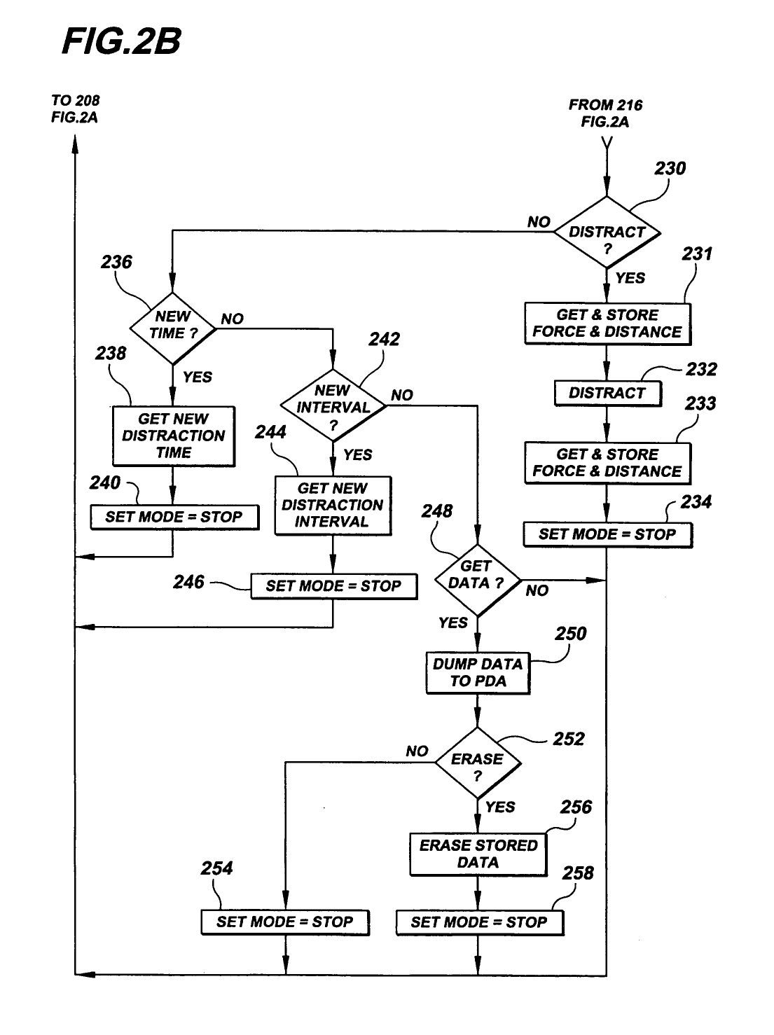

One example of the programming for the microcontroller will now be described with reference to the flow diagram 200 of FIGS. 2A and 2B. Upon power on of the microcontroller 102, the processor is initialized (Step 202), default control values are loaded (Step 204), and a wait period of approximately 10 seconds is entered (Step 206). After the wait period, the microcontroller checks for any commands from the wireless communications module 114 (Step 208). An inquiry is made as to whether a “stop” command was received, indicating to stop distracting (Inquiry 210). If so, all actions are stopped (Step 212), and, after a short wait period of about one second (Step 214), the program loops back to check communications (Step 208).

If a command to stop distracting is not received (Step 210), then an inquiry is made as to whether a “start” command was received from the wireless communications module, indicating to begin a full distraction (Step 216). If so, then the microcontroller retrieves and stores the current displacement measurement from displacement sensor 108 and the current force measurement from force sensor 110 via analog circuitry 112 (Step 218). After retrieving and storing the force and displacement measurements, the microcontroller initiates a distraction by sending a signal to SMA switch 106 (Step 220). After the distraction is complete, the force and displacement measurements are again retrieved and stored (Step 222). After retrieving and storing post-distraction force and displacement measurements, an extended wait period of approximately 12 seconds is entered (Step 224). After the wait period, communications are again checked (Step 225), and an inquiry is made as to whether a new command was received (Step 226). If a new command was received, the program loops back to Step 210. If a new command was not received, an inquiry is made as to whether to engage in another full distraction (Inquiry 228). If not, the program loops back to the wait period of Step 224. If another distraction is called for, the program loops back to Step 218.

Returning now to Step 216, if a command to start a full distraction was not received, an inquiry is made as to whether a “distract now” command was received, indicating to perform an immediate distraction (Inquiry 230). If so, then the microcontroller retrieves and stores the current displacement measurement from displacement sensor 108 and the current force measurement from force sensor 110 via analog circuitry 112 (Step 231). After receiving and storing the force and displacement measurements, the microcontroller initiates a distraction by sending a signal to SMA switch 106 (Step 232). After the distraction is complete, the force and displacement measurements are again retrieved and stored (Step 233), the command mode is set to stop (Step 234), and the program loops back to Step 208.

If a “distract” command, indicating to perform an immediate distraction, was not received (Inquiry 230), an inquiry is made as to whether a “new time” command was received, indicating to obtain a new distraction time (Inquiry 236). If a new distraction time is to be obtained, it is then obtained (Step 238), all distractions are stopped (Step 240), and the program returns to Step 208 to check communications.

If a “new time” command was not received (Inquiry 236), then an inquiry is made as to whether a “new interval” command was received, indicating to obtain a new distraction interval (Inquiry 242). If a new distraction interval is to be obtained, it is then obtained (Step 244), and all distractions are stopped (Step 246). The program then returns to Step 208.

If a “new interval” command was not received (Inquiry 242), then an inquiry is made as to whether a “get data” command was received, indicating to send the stored displacement and force sensor measurements to the outside world via communications module 114 (Inquiry 248). If a “get data” command was received, the stored data is then sent (Step 250). In the present example, the data is received by a personal digital assistant. If no “get data” command was received, then the program returns to Step 208.

After sending the stored data in Step 250, an inquiry is made as to whether a command was received to erase the stored force and displacement measurements (Inquiry 252). If not, all distractions are stopped (Step 254), and the program returns to Step 208. If the stored data is to be erased, then it is erased (Step 256), all distractions are stopped (Step 258), and the program returns to Step 208 to check communications.

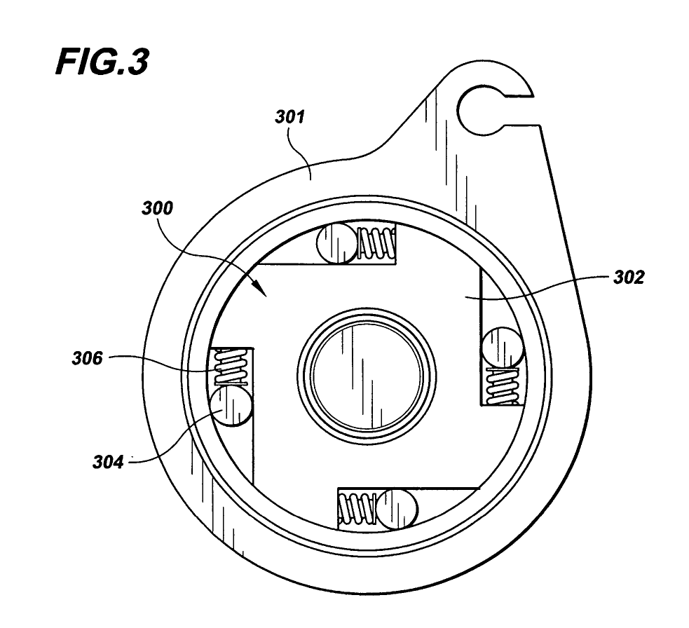

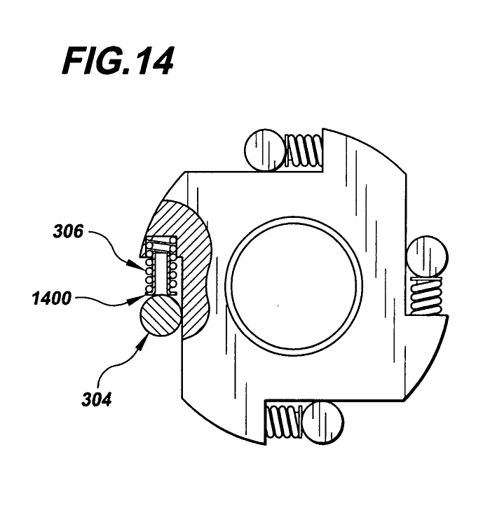

FIG. 3 is a cross-sectional view of one example of the SMA tensioner 118 in detail. The tensioner drives the distractions while minimizing backlash. In the presently preferred embodiment, the tensioner comprises two one-way roller clutches, e.g., clutch 300. One commercially available example of a one-way roller clutch is the internal portion of the TINY-CLUTCH available from Helander Products, Inc., Clinton, Conn. Clutch 300 is shown in housing 301, and comprises a rotor/cam 302, rollers (e.g., roller 304), springs (e.g., spring 306), and bushings (e.g., bushing 1400 best shown in FIG. 14).

Although clutch 300 is presently preferred, other one-way clutches could be used. Of course, any clutch used will need to be of a size that is acceptable for the application. For use with bone distraction, the clutch should have as little backlash as possible, zero or near zero preferably. As one skilled in the art will know, backlash is the amount of play between the main movable members in a gear or clutch, in this case, between the housing and the cam.

Prior to describing the operation of clutch 300, a general overview of the operation of a shape memory allow will now be provided. The SMA Actuator takes advantage of two shape-memory properties for its operation: ease of deforming the SMA below its transition temperature, and the ability to return to its pre-deformed shape upon heating above its transition temperature. These characteristics and their physical basis are discussed below with respect to Nitinol, one example of a SMA useful with the present invention. Nitinol is an alloy of nickel and titanium. One example of a commercially available Nitinol wire is FLEXINOL, available from Dynalloy Inc. of Costa Mesa, Calif.

Above the transition temperature, the Nitinol microstructure is in an austenitic phase. The austenitic phase is a body-centered cubic (bcc) phase with 90 degrees between each primary crystal axis. This bcc phase is actually composed of two intermeshed cubic lattice structures, one with titanium atoms at the cubic lattice points, and one with nickel atoms at the lattice locations. The cubic titanium structure is displaced from the nickel cubic structure to form the bbc structure, and consequently, each nickel atom is at the center of a cube with titanium atoms at its corners, and, similarly, each titanium atom is at the center of a cube having nickel atoms at its corners.

Below the transition temperature, the Nitinol microstructure is in a martensitic phase. This phase is similar in atomic arrangement to the austenitic phase described above, but with a monoclinic structure rather than a cubic structure, with the angle between the two oblique axes of the monoclinic structure, close to (but not equal to) 90 degrees.

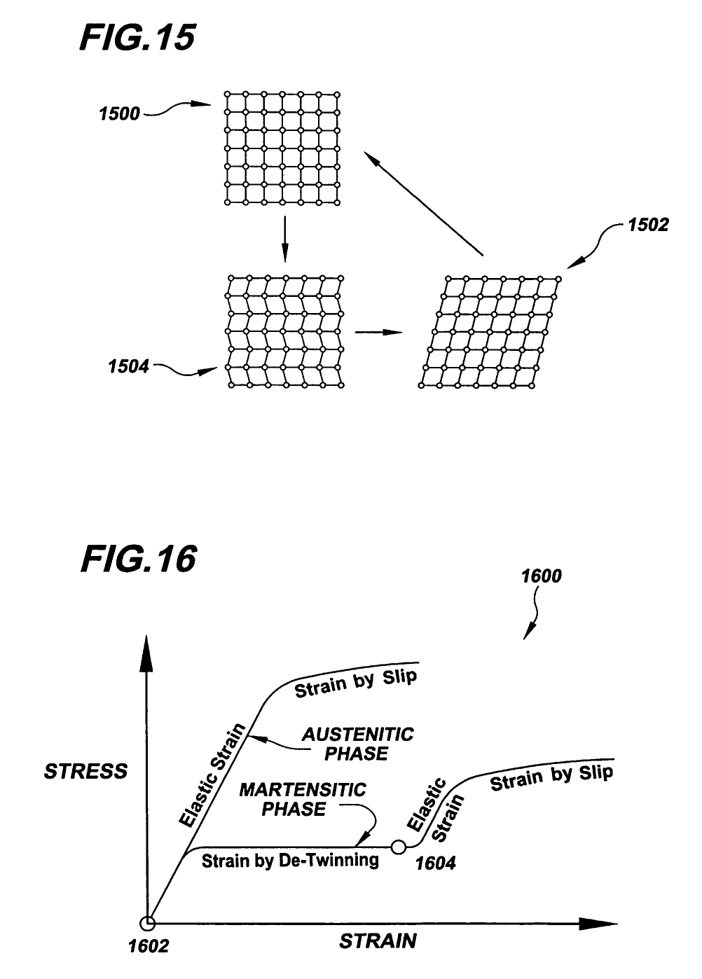

The austenitic and martensitic structures can be shown schematically in two-dimensional form as structures 1500 and 1502, respectively, in FIG. 15. Because of its cubic structure, deformation of the austenitic phase shown by structure 1500 can only occur by slippage of one atomic place relative to another. This slippage results in the moving of atoms from lattice point to lattice point so that the identity of neighboring atoms after the slippage changes. On the other hand, because of the monoclinic structure, the martensitic phase can deform by either slipping or by “twinning.” Twinning is a motion of crystal planes relative to one another that results in strain without the motion of atoms from lattice point to lattice point and without a change in the identity of neighboring atoms. In twinning, the atoms on both sides of a twinning place appear as mirror images of each other. In microstructure 1504 in FIG. 15, every horizontal plane is a twinning plane, and the crystal is said to be fully twinned.

Transition from microstructure 1504 to 1502 (and vice versa) of FIG. 15 can be accomplished without slip, and occurs quite easily in the Nitinol martensitic phase. This accounts for the softness and ease of deformation of Nitinol in its martensitic phase. It is also responsible for the fact that very large deformations (as large as 8% strain) can occur before the structure is fully detwinned, and further strain can only occur by slip.

When Nitinol is cooled from a temperature above its phase transition temperature to a temperature below its phase transition, the low temperature martensitic phase is physically constrained during its formation by the surrounding, as yet, untransformed austenite. Consequently, the austenitic structure transforms into a martensitic structure with a shape similar to the shape of the original austenitic structure, that is, the rectangular austenitic structure 1500 in FIG. 15 transforms to the rectangular (and hence fully twinned) martensitic structure 1504. Straining this fully twinned martensitic phase results in easy transition to a more detwinned structure (e.g., the fully detwinned martensitic phase 1502). This deformation is referred to as super-elastic deformation because, though it is relatively large, it occurs without the slippage of atoms relative to each other. Because the identity of neighboring atoms has not been changed by crystal plane slipping during this strain, reheating of structure 1502 above the transformation temperature causes the resulting austenitic microstructure to revert to the original rectangular shape, that is, the structure reverts from the deformed structure 1502 back to un-deformed structure 1500. This behavior forms the basis for shape memory.

FIG. 16 is a graph 1600 illustrating, in idealized fashion, the effect of the above on the stress-strain characteristics of the austenitic and martensitic Nitinol phases. Since the cubic austenitic structure is constrained to yield plastically by slip, the austenitic phase is relatively strong and hard with a typical yield strength of 120 ksi, and a typical ultimate strength of 155 ksi. The martensitic phase, on the other hand, is softer and weaker and can elastically strain by detwinning at stress levels that are typically as low as 20 ksi and can strain by detwinning to non-slip strains as high as 8%. The locations of the microstructures 1500 and 1504 from FIG. 15 are shown schematically in FIG. 16 by the corresponding point 1602, while microstructure 1502 is shown at point 1604.

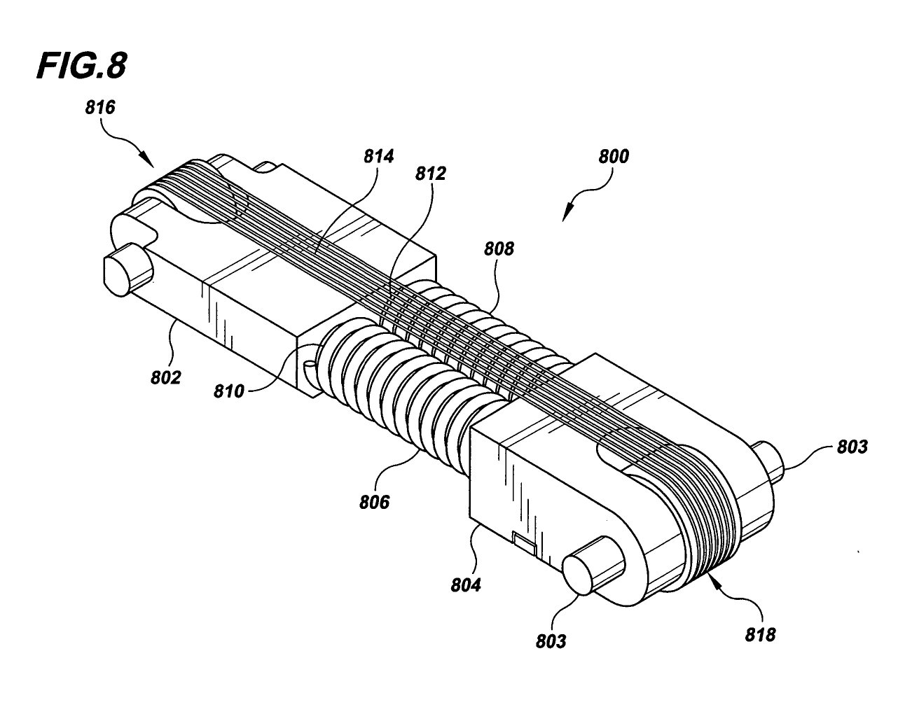

Operation of clutch 300 in the context of the distraction device will now be described in detail. Activation of the SMA actuator applies DC voltage to the SMA. Voltage is applied to the SMA by activating switch 106 shown in FIG. 1. In one example, the switch comprises dual power MOSFETs (Metal Oxide Semiconductor Field Effect Transistor) connected in parallel and electrically coupled to microcontroller 102. Activation of the switch allows power from power source 120, electrically coupled to the SMA switch, to flow to SMA actuator 104. Deactivation occurs by removing the DC voltage applied to the SMA, i.e., switch 106 is turned off. This allows the SMA to cool to below transition temperature and revert to the low-temperature phase so that it can be again super-elastically re-extended by the bias springs (see description of FIG. 8).

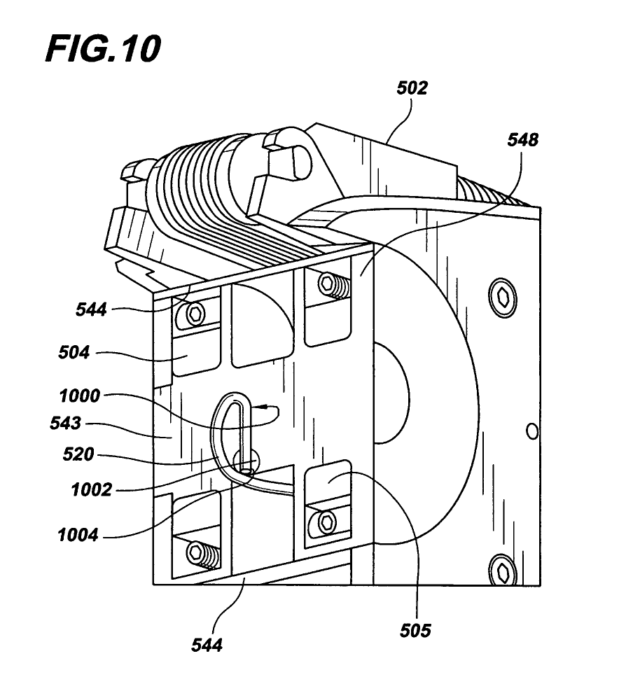

FIG. 10 is a cut-away view of the back end 540 of the distraction device 500 shown in FIG. 5 and described subsequently. As noted above, minimizing backlash is a goal when a clutch-based SMA tensioner is used. In the present example, the goal is achieved through the use of two clutches. A driving clutch 504 is attached to rotor/shaft 543, and is housed within the bore of a swing arm 544, which is driven (rotated) by SMA actuator 502. A holding clutch 505 is also attached to rotor/shaft 543, but is housed within a stationary bushing 548. The holding clutch prevents counter-rotation when the SMA actuator is relaxing after a distraction, thereby minimizing backlash.

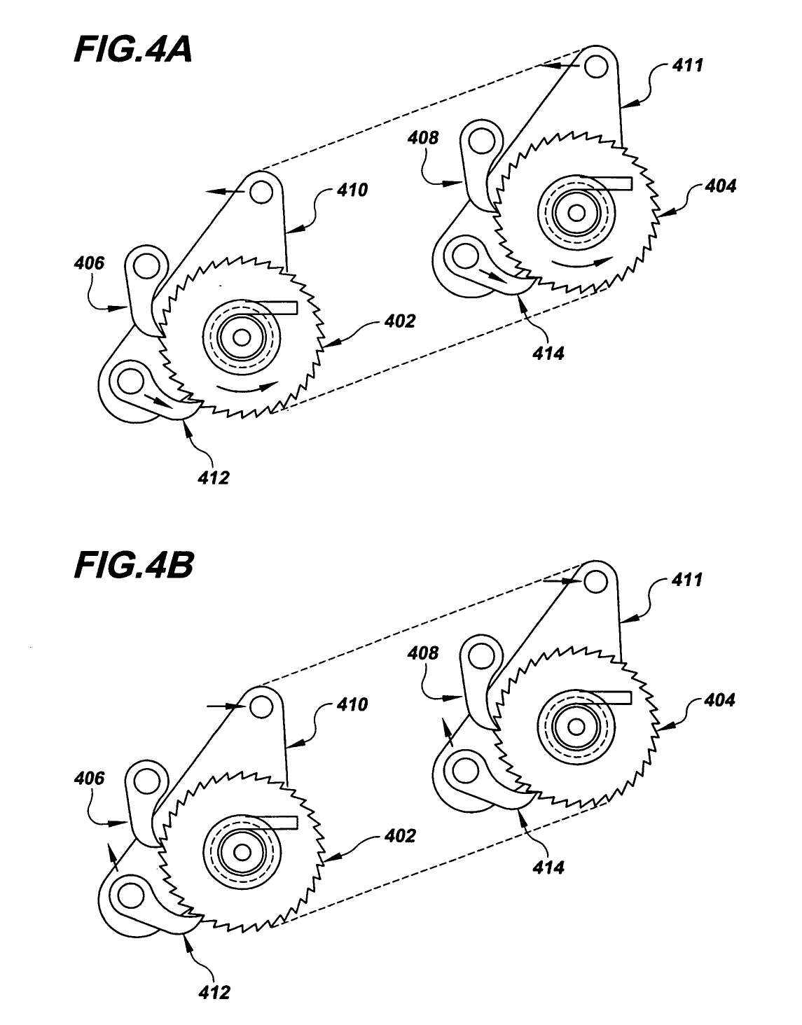

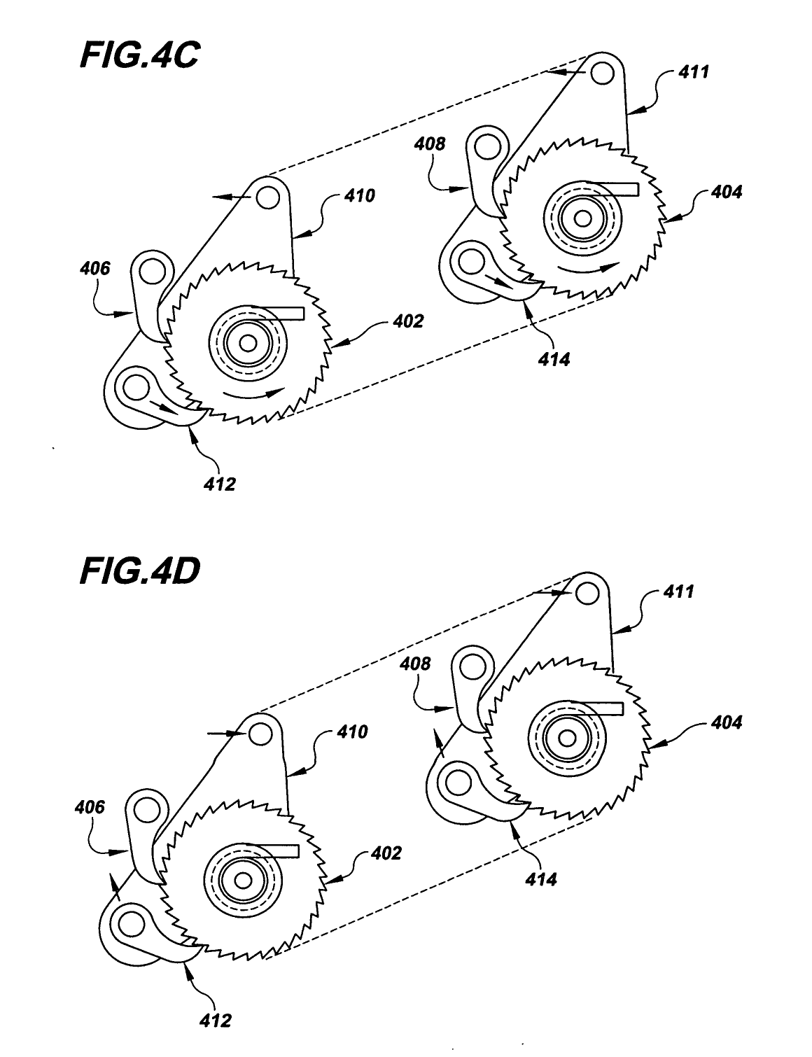

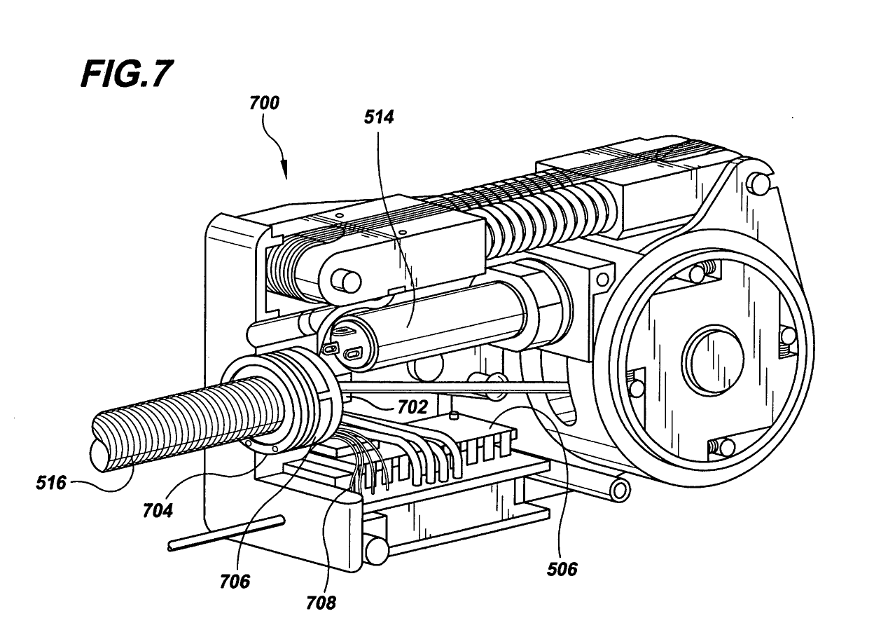

FIGS. 4A-4D depict another example of the SMA tensioner 118 in detail. The tensioner in this example takes the form of a ratchet and pawl system 400, shown in various stages of operation. The ratchet and pawl system comprises two ratchet wheels 402 and 404, a set of two holding pawls 406 and 408, two drive arms 410 and 411, and a set of two drive pawls 412 and 414. The key to proper operation of the two ratchet wheel, four pawl mechanism is to arrange the wheels and pawls so that the two ratchet wheels operate sequentially relative to one another in “leap frog” fashion. One way to accomplish this is to locate the drive and holding pawls at the same angular location relative to each other, but to angularly displace the two ratchet wheels by half a tooth spacing relative to each other. The operation of the two ratchet wheel, four pawl mechanism will now be described in detail. For reasons of illustration clarity, the two ratchet wheels (which operate on the same axis of rotation in the actual mechanism) are shown with their axis of rotation displaced from each other. They fit into the same space in the distraction device and with the same orientation as the one-way roller clutches shown, for example, in FIGS. 5 and 7.

FIG. 4A depicts the operation of two ratchet, four pawl mechanism when the SMA actuator is contracting (activated) and pulling on its drive arm attachment. For illustration, consider FIG. 7 with the SMA actuator contracting, pulling the drive arms to the left and causing them to rotate in the counter-clockwise (CCW) direction. Drive pawl 412 is engaged with ratchet wheel 402, causing it to rotate in the CCW direction. The CCW rotation of the two ratchet wheels continues until holding pawl 408 slides over the tooth and slips into a holding position to prevent clockwise (CW) rotation of the ratchet wheel mechanism when the SMA actuator reextends (deactivates). This next step is illustrated in FIG. 4B.

FIG. 4B depicts the ratchet and pawl system with the SMA actuator deactivated and holding pawl 408 holding the ratchet wheel assembly against the distraction cable load and preventing it from rotating in the CW direction and unwinding the cable. As the SMA actuator reextends, it causes drive arms 410 and 411 to rotate in the CW direction, allowing drive pawl 414 to ride up on and over the tooth and slip behind it into drive position.

FIG. 4C depicts the operation of the mechanism during the next distraction increment. Now drive pawl 414 is engaged on ratchet wheel 404 and driving both ratchet wheels in the CCW direction. The ratchet wheels again rotate until holding pawl 406 slides up on, and falls behind the next tooth on ratchet wheel 402, as shown in FIG. 4D.

FIG. 4D shows the two ratchet, four pawl mechanism with the SMA actuator deactivated and holding pawl 406 holding the ratchet wheel assembly against the distraction cable load and preventing it from rotating in the CW direction and unwinding the cable. As the SMA actuator reextends, it causes the drive arms to rotate in the CW direction, allowing drive pawl 412 to ride up on and over the tooth and slip behind it into drive position. The mechanism has now returned to a condition such that the next SMA activation will begin to repeat the sequence of FIGS. 4A-4D.

Of course, it will be understood that for some applications, a design with a different number of ratchet wheels (fewer or more) may be called for. The number of ratchet wheels in this example was selected to strike a balance between the minimum distraction increment (tooth size) and the maximum distraction load (tooth strength).

FIG. 5 depicts an open-housing view of one example of a distraction device 500 in accordance with the present invention. Shown is the SMA actuator 502, two one-way roller clutches 504 and 505 (505 shown in FIG. 10), microcontroller 506, RF aerial antenna 508, cable system 510 to a bone (not shown) under distraction, backbone 512, and displacement sensor 514. It should be noted that the bone and connection thereto are not shown, as they form no part of the present invention.

Cable system 510 comprises a first sheath 518 that surrounds distraction cable 520, at the bone end, and a second sheath 516 surrounding the distraction cable at the distraction device end. The distraction cable comprises, for example, a braided chromium-cobalt cable, and is coupled to rotor/shaft 543 by feeding the same through an opening 1000 (see FIG. 10) therein. A ball 1002, for example, a ⅛ inch diameter stainless steel ball, is soldered at one end 1004 of the distraction cable to hold the cable to the rotor/shaft, and ensure it is wound around the rotor/shaft as distractions proceed. A cap 522 transmits force from the smaller to the larger sheath and holds them together.

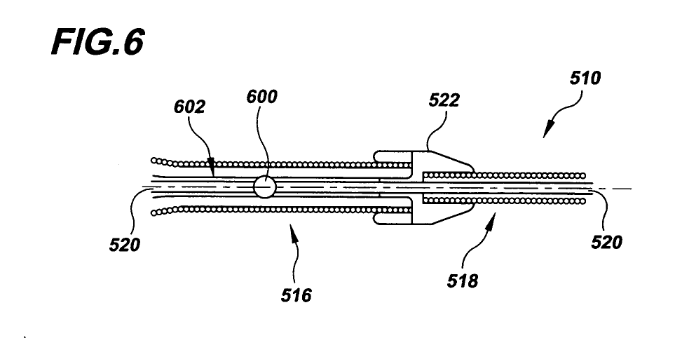

FIG. 6 is a cross-sectional view of cable system 510 from FIG. 5 taken along lines 6-6. Shown is an in-line sealing ball 600 attached to distraction cable 520. As the distraction cable is drawn into the distraction device, the ball slides along the inner diameter of sealing tube 602, and effectively seals out any fluids as the cable is drawn into the distraction device.

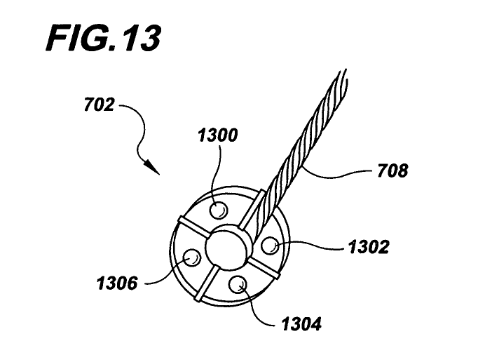

FIG. 7 is a cut-away view of a portion 700 of the distraction device 500 of FIG. 5, more clearly showing force sensor 702. A retaining ring 704 and O-ring 706 seal the sensor and other internal components from the body of the patient. Force is transferred from sheath 516 through the force sensor 702 and finally supported by the backbone 512 (shown in FIG. 5). Backbone 512 covers the force sensor and supports the reactionary load from the cable that is pulling on the bone. In addition, the backbone in the present example also supports the side plates of the device housing, one end of the SMA actuator, and reactionary loads from the SMA actuator during compression. The force sensor communicates with microcontroller 506 over wires 708. Shown more fully in FIG. 13, is one example of force sensor 702. Sensor 702 comprises, for example, a “washer” style load cell with strain gauges 1300, 1302, 1304 and 1306 in a bridge arrangement. In the present example, the force sensor can sense zero to about 300 lbf compression with an excitation voltage of about 5 VDC and output of about 1 mV/V and an accuracy of at least about 1%.

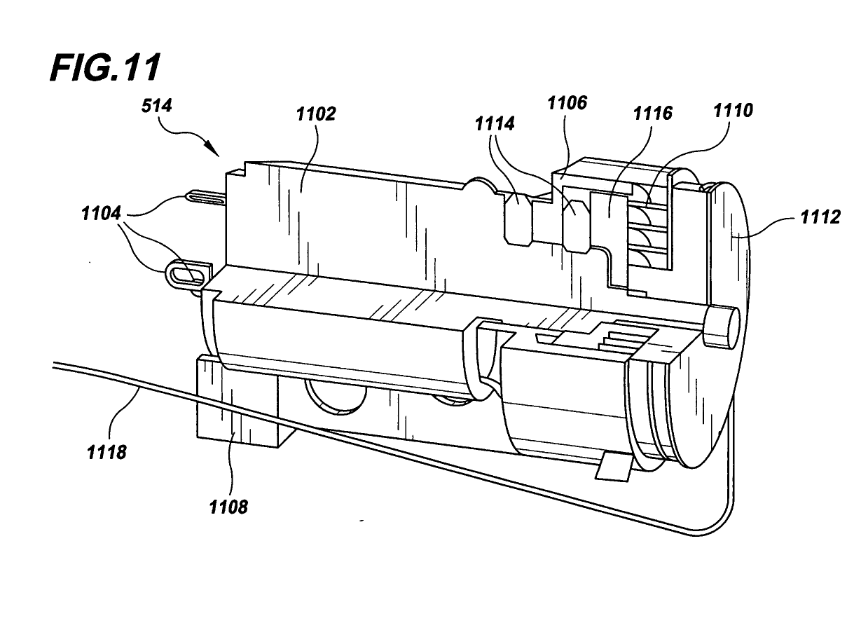

FIG. 11 is a more detailed, cut-away view of the displacement sensor 514 shown in FIG. 7. The displacement sensor comprises a miniature, three-turn potentiometer 1102 with electrical connectors 1104 at a first end. Connectors 1104 are electrically coupled to microcontroller 506, and provide a voltage from which the microcontroller determines a resistance value R. A displacement value X can then be determined in accordance with the following relationship:

$$X = {X}_{out} + [ \frac{( {X}_{i n} - {X}_{out} )}{( {R}_{i n} - {R}_{out} )} * ( R - {R}_{out} ) ]$$

where Rₒᵤₜ is the resistance of potentiometer 1102 when the cable displacement is Xₒᵤₜ, and Rᵢₙ is the resistance when the cable displacement is Xᵢₙ. The displacement value may be calculated, for example, by the microcontroller, manually, or in an automated fashion (e.g., a computer) outside the distraction device after obtaining the resistance data through the wireless communications module, described more fully herein.

The other end of the sensor comprises a housing 1106 coupled to a bracket 1108 for connecting to the housing of the distraction device. Coupled to housing 1106 is a wind-up or power-spring 1110 and wind-up reel or spool 1112. The components are held together with nuts 1114, and a spacer 1116 is present. Wrapped around the wind-up reel or spool is a cable 1118 that is coupled to the distraction cable for measuring displacement from a distraction.

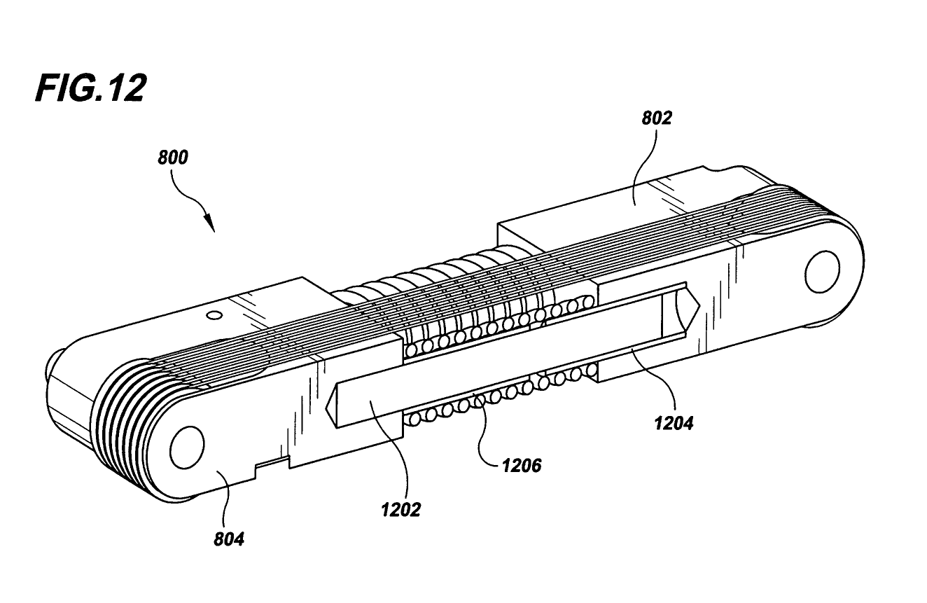

FIG. 8 depicts one example of the SMA actuator 800 (104 in FIG. 1). The actuator has a block-and-tackle design, comprising two blocks 802 and 804. Block 804 is fixed to the housing of the distraction device at either end of a pin 803, while block 804 is coupled to the SMA tensioner (118 in FIG. 1). Between the two blocks are two bias springs 806 and 808 for maintaining tension and covering spring guides 810 and 812. Block 802 is coupled to swing arm 544 as best shown in FIG. 10, allowing it to telescope. As shown in FIG. 12, the spring guides each comprise a rod 1202 coupled to block 804, and a surrounding sleeve 1204 coupled to block 802. A second sleeve 1206 floating between the blocks restrains the radial deflection of the springs when compressed during distraction. SMA wire 814 connects the blocks, and is wrapped around seven pulleys 816 in block 802, and seven pulleys 818 in block 804. Prior to assembly on the pulleys, the SMA wire is stretched axially to ensure that it is in its fully detwinned state (State 1502 in FIG. 4) after which assembly of the SMA actuator proceeds. Tension in the seven turns of SMA wire serves to maintain the axial separation of the blocks against the bias spring separation force. At the same time, the wire tension induced by this force serves to maintain the wire in the fully detwinned state (1502 in FIG. 15).

The SMA actuator can be in one of two states: activated and deactivated. In the activated state, the SMA wire in the SMA actuator is coupling, via the SMA switch, to the power source so that an electric current passes through the wire. The resultant Joule heating of the wire by this electric current raises the wire temperature and transitions the wire into its austenitic state. In the deactivated state, the SMA switch decouples the SMA wire from the power source so that it cools (by convective heat transfer to the surrounding air) to below its transition temperature and reverts to its martensitic state.

In the deactivated state, with the SMA wire in its relative weak martensitic state, the tension stress applied to the wire by the bias spring causes the SMA wire to strain by detwinning until it is almost fully detwinned. In the activated state, with the wire in its undeformed and relatively strong austenitic state, the removal of the detwinning deformation causes the wire to contract and further compress the bias spring.

As a result, activating the SMA actuator causes it to forcibly shorten as the wire transitions to its austenitic state, while deactivating the engine causes the engine to reextend as the wire reverts to its martensitic state and is detwinned in response to the bias spring induced wire tension.

In the present example, seven turns of 0.015 inch diameter FLEXINOL wire was used. Of course, the wire diameter, the number of wire turns, and length of wire spanning the distance between the pulleys 816 and 818 will depend on the particular application. For example, the number of turns and the wire diameter will determine the maximum wire stress experienced by the wire under the maximum distraction load. Excessive stress will lead to early fatigue failure of the actuator before the number of activations required to achieve the full cable distraction. Too short a distance between the pulleys results in insufficient wire contraction to achieve the required level of distraction per activation, while too many turns increases the amount of wire that must be heated per actuation and limits the number of actuations that can be obtained from a given power source.

Returning to FIG. 1, the power source 120 chosen will depend on the particular application. However, in general, the criteria to consider for a power source useful with the distraction device of the present invention comprises size, output, capacity, internal resistance, and cost. Depending on the type of power source, there may also be additional or different criteria to consider. One example of a power source useful with the distraction device of the present invention is a battery, though other types may instead be used, such as, for example, fuel cells. Since the distraction device is implantable, the size of the of the power source is an obvious concern. Preferably, the power source is as compact as possible, though size will usually be weighed against the other criteria. The output must be enough to provide the power necessary for a given distraction. In the present example, the output needs to be enough to force a change of state in the SMA wire, as well as move the SMA tensioner under load. The capacity (mA-hr) of the power source needs to be sufficient for the expected time frame to accomplish the distraction goal. Since distraction typically involves a high current draw, the power source cannot have too high an internal resistance. Note that batteries, for example, connected in series have a higher internal resistance. Finally, power sources have a wide range of costs, depending in large part on the technology used.

Preferably, a battery is used as the power source, and most preferably, a lithium sulfur dioxide battery. One example of such a commercially available battery is model LO35SX from Saft America, Inc., located in Valdese, N.C., which is rated at 2,000 mA-hr in capacity. These batteries are about ⅔ the length of a standard C cell alkaline battery and about the same width.

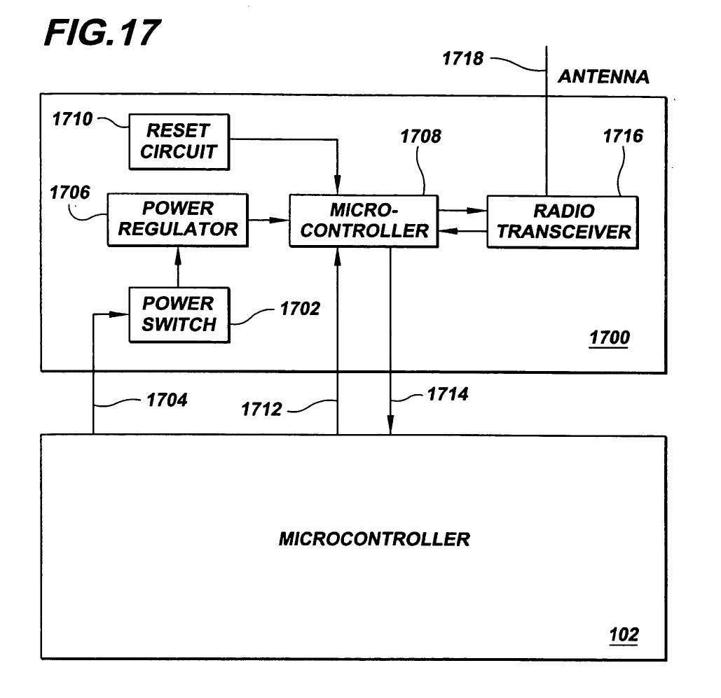

Wireless communications module 114 in FIG. 1 can take different forms. In one example shown in FIG. 17, the communications module takes the form of a radio transceiver module 1700. The transceiver is a bi-directional data communications radio for communications between an implanted device and an external monitor or controller. In the U.S., the transceiver operates in the U.S. FCC Medical Implant Communications Band, currently 402-405 MHz and a maximum radiated power of 25 microwatts. Although the basic component design of the transceiver is conventional, it has been sized for the application.

Briefly, the transceiver module comprises a power switch 1702 that receives a signal over line 1704 from microcontroller 102 to apply DC power from the microcontroller to a power regulator 1706, which stabilizes and conditions the DC power used by microcontroller 1708. When power is applied by the switch, a reset circuit 1710 holds microcontroller 1708 in a reset state until the power is stabilized. Microcontroller 1708 handles all transmit 1712 and receive 1714 communications between microcontroller 102 and radio transceiver 1716. Microcontroller 1708 also arbitrates the hardware handshaking between the two microcontrollers and transfers data to and from the radio transceiver, which converts data to (and from) an FM signal for broadcasting via antenna 1718.

In another example, the wireless communications module takes the form of an infrared transceiver. As one skilled in the art will know, an infrared transceiver comprises a transmitting diode and a receiving phototransistor operating in the infrared region. They are usually matched in size and in wavelength. One example of a commercially available infrared transceiver is the QED122 Infrared Light Emitting Diode and the QSD122 Infrared Phototransistor, both from Fairchild Semiconductor in Portland, Me. Another example is the Fairchild QEB373 Subminiature Infrared Emitting Diode and Fairchild QSB363 Subminiature Infrared Phototransistor. Both pair operate at a peak emissions wavelength (transmitter) and peak sensitivity (receiver) of 880 nm.

In either embodiment, it should be understood that the communications module could be just a receiver or a transmitter. For example, if no data is to be sent out, then a receiver to receive commands and/or programming would be enough. As another example, if the microcontroller is not to be programmable, but data is desired for monitoring, then a transmitter is appropriate.

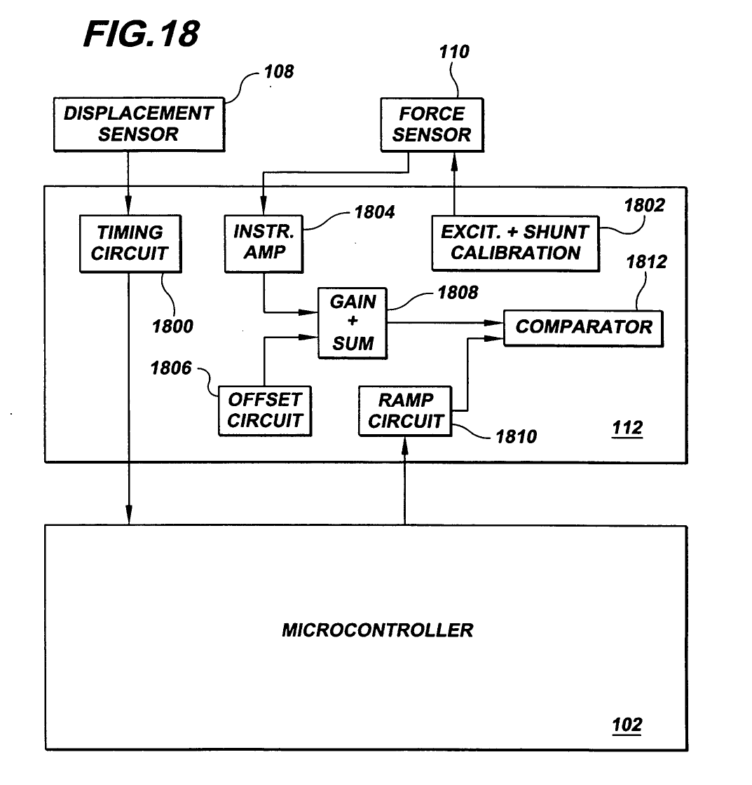

FIG. 18 is a block diagram of one example of the analog circuitry 112 of FIG. 1. A timing circuit 1800 is used by microcontroller 102 to measure the resistance of displacement sensor 108. This is done by charging a known capacitance through the displacement sensor, and tracking the time to reach a predetermined threshold. This yields an indirect measurement of the sensor resistance. To make a resistance measurement, the microprocessor first discharges the capacitor. Then the time to charge the capacitor to approximately 1.5 volts DC through the displacement sensor is measured in 2 microsecond increments. The resistance can then be calculated from the number of increments with the equation below.
R(KΩ)=(increments)÷(600×C(μf))
The excitation and shunt calibration network 1802 is used to power the force sensor 110 and to linearize its voltage output over the usable range. The instrumentation amplifier 1804 is used to make a differential voltage measurement across the force sensor and convert this reading to a single ended signal. The offset circuit 1806 produces a fixed, known voltage value for the gain and summing circuit 1808. The gain and summing circuit adds the output of the offset circuit to the output of the instrumentation amplifier and provides additional force signal gain. The ramp circuit 1810 smoothes a pulse width modulated output from the microcontroller and buffers this signal to produce an increasing 256 step voltage waveform. The output of the comparator circuit 1812 switches a digital input to the microcontroller from a logical zero to a logical one when the ramp circuit output equals the conditioned output of the force sensor. The value of the pulse width modulation output of the microcontroller at the time the comparator switches may then be scaled in software to equal the voltage of the force sensor output.

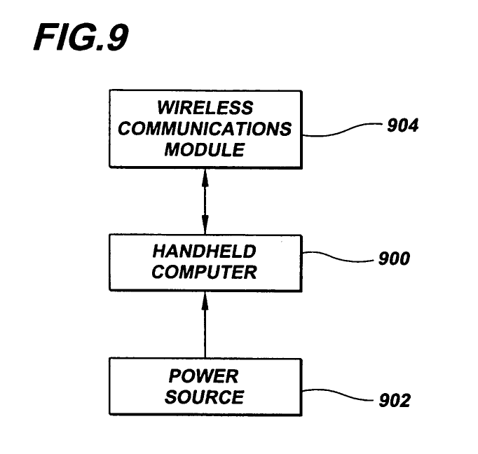

FIG. 9 is a block diagram of one example of a device that can wirelessly communicate with the bone distraction device. The device comprises a handheld computer 900, power source 902, and wireless communications module 904. One example of a handheld computer is a personal digital assistant (PDA) running either the PALM operating system or WINDOWS MOBILE operating system. The power source could be, for example, an AC power supply or a battery. One example of a battery is a lithium ion rechargeable battery. The wireless communications module takes a form to match that employed by the implantable distraction device. For example, it can take the form of a radio transmitter, receiver or transceiver, or, as another example, an infrared transmitter, receiver or transceiver. Many PDA's currently available include integrated infrared transceivers. Of course, the wireless communications module could also take the form of an add-on card or device, for example, a card designed to fit into a flash memory slot in the PDA. Such devices are commercially available and, other than communicating wirelessly with the implantable bone distraction device, form no part of the present invention, in and of themselves.

One example of the operation of handheld computer 900 to communicate with the distraction device 100 of FIG. 1 will now be described. The operation would, for example, be governed by a computer program written for the handheld computer. Handheld computer 900 is initially set with the communication addresses of wireless communication modules 904 and 114. A communications link is then established, with an error message if no link can be established. Once the communications link is established, any command options chosen to be included could be selected. For example, commands to stop distractions in progress, start a distraction and change the time interval between distractions could be included. In addition, commands regarding the optional sensors could be included, for example, acquiring data from a given sensor, as well as communicating the data to another computer.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A bone distraction device, comprising:
a distraction driver for incrementally distracting bone and minimizing backlash;
an actuator coupled to the distraction driver for actuating the distraction driver; and
a microcontroller electrically coupled to the actuator for controlling the actuator;
at least one of a wireless communications receiver electrically coupled to the microcontroller for receiving information and a wireless communications transmitter electrically coupled to the microcontroller for transmitting information;
wherein the bone distraction device is implantable.

2. The bone distraction device of claim 1, wherein the distraction driver comprises a plurality of clutches.

3. The bone distraction device of claim 2, wherein the plurality of clutches comprises a drive clutch and a holding clutch.

4. The bone distraction device of claim 3, wherein the drive clutch and the holding clutch each comprises a one-way roller clutch.

5. The bone distraction device of claim 1, wherein the distraction driver comprises a plurality of ratchets and a plurality of pawls.

6. The bone distraction device of claim 5, wherein the plurality of ratchets is arranged to operate sequentially relative to one another, and wherein the plurality of pawls comprises a holding pawl and a drive pawl for each of the plurality of ratchets.

7. The bone distraction device of claim 1, wherein the actuator comprises a shape memory alloy for causing a distraction by the distraction driver.

8. The bone distraction device of claim 7, further comprising a housing for the bone distraction device, wherein the actuator comprises:
a pair of members coupled together by the shape memory alloy, one of the members being coupled to the distraction driver and the other of the pair of members being coupled to the housing, wherein the shape memory alloy tends to pull the members together when activated;
one or more spring members situated between the members tending to push the members away from one another.

9. The bone distraction device of claim 8, wherein the shape memory alloy comprises a wire, and wherein the wire is wound around the pair of members.

10. The bone distraction device of claim 7, further comprising a switch electrically coupled between the actuator and the microcontroller for controlling actuation of the shape memory alloy.

11. The bone distraction device of claim 1, wherein the at least one of a wireless communications receiver and a wireless communications transmitter comprises at least one of an infrared receiver and an infrared transmitter.

12. The bone distraction device of claim 1, wherein the at least one of a wireless communications receiver and a wireless communications transmitter comprises at least one of a radio frequency receiver and a radio frequency transmitter.

13. The bone distraction device of claim 1, further comprising a displacement sensor for sensing displacement caused by a distraction.

14. The bone distraction device of claim 13, further comprising a distraction cable coupled to the distraction driver, wherein the displacement sensor is coupled to the distraction cable and comprises a potentiometer electrically coupled to the microcontroller for providing voltage information from which resistance and displacement can be determined.

15. The bone distraction device of claim 1, further comprising a force sensor for sensing the force being applied by a distraction.

16. The bone distraction device of claim 15, further comprising a sheath for covering a distraction cable, wherein the force sensor is coupled to the sheath and comprises a washer-style load cell having a plurality of strain gauges.

17. The bone distraction device of claim 1, further comprising a distraction cable coupled to the distraction driver, wherein the distraction cable is sealed against body fluids.

18. The bone distraction device of claim 1, wherein the at least one of a wireless communications receiver, and a wireless communications transmitter comprises a wireless communications receiver, and wherein the microcontroller is controllable via signals received from the wireless communications receiver.

19. The bone distraction device of claim 18, wherein the information comprises at least one of an immediate distraction command and a stop-distraction command.

20. The bone distraction device of claim 1, further comprising a sensor electrically coupled to the microcontroller for sensing a characteristic of a distraction and providing to the microcontroller, wherein the at least one of a wireless communications receiver and a wireless communications transmitter comprises a wireless communications transmitter, and wherein the information comprises information regarding the characteristic.

21. The bone distraction device of claim 20, wherein the at least one of a wireless communications receiver and a wireless communications transmitter further comprises a wireless communications receiver, and wherein the information comprises at least one command to obtain and transmit the information regarding the characteristic.

22. The bone distraction device of claim 1, wherein the at least one of a wireless communications receiver and a wireless communications transmitter comprises a wireless communications receiver, wherein the microcontroller is programmable, and wherein the information comprises microcontroller programming information.

23. The bone distraction device of claim 22, wherein the microcontroller programming information comprises a distraction time interval.

24. The bone distraction device of claim 22, wherein the microcontroller programming information comprises a distraction length.

25. The bone distraction device of claim 1, further comprising a battery therefor.

26. The bone distraction device of claim 1, wherein the at least one of a wireless communications receiver and a wireless communications transmitter comprises a wireless communications transceiver.

27. A system for bone distraction, comprising:
a bone distraction device, comprising:
a distraction driver for incrementally distracting bone and minimizing backlash;
an actuator coupled to the distraction driver for actuating the distraction driver using a shape memory alloy;
a microcontroller electrically coupled to the actuator for controlling the actuator; and
a wireless communications transceiver electrically coupled to the microcontroller for transmitting and receiving information;
wherein the bone distraction device is implantable; and
a wireless communications device for transmitting information to and receiving information from the wireless communications transceiver.

28. The system of claim 27, wherein the wireless communications device comprises a handheld computing device.

29. The system of claim 27, wherein the distraction driver comprises a plurality of one-way roller clutches.

30. The system of claim 27, further comprising at least one sensor electrically coupled to the microcontroller for sensing at least one characteristic of a distraction and providing to the microcontroller, wherein the information comprises information regarding the at least one characteristic.

31. The system of claim 27, the bone distraction device further comprising a switch electrically coupled between the actuator and the microcontroller for controlling activation of the shape memory alloy.
